(19) United States
(12) Patent Application Publication
(10) Pub. No.: US 2026/0115384 A1
(43) Pub. Date: Apr. 30, 2026

(54) INFUSION DEVICE AND ASSOCIATED SYSTEMS AND METHODS

(71) Applicant: MEDTRONIC MINIMED, INC., Northridge, CA (US)

(72) Inventors: David C. Antonio, Montrose, CA (US); Elden Cartwright, Burbank, CA (US); Eric A. Larson, Simi Valley, CA (US)

(21) Appl. No.: 19/332,763

(22) Filed: Sep. 18, 2025

Related U.S. Application Data
(60) Provisional application No. 63/712,317, filed on Oct. 25, 2024.

(51) Int. Cl. A61M 5/172; A61M 5/142

(52) U.S. Cl. CPC A61M 5/1723; A61M 5/14248; A61M 2005/14268

(57) ABSTRACT

A medical device in accordance with an embodiment of the present technology includes a first component and a second component configured to be detachably coupled to the first component. The first component can include a first housing with a recessed portion and a boss. The second component can comprise a second housing configured to be received within the recessed portion of the first housing while the first and second components are detachably coupled to one another. The second housing can define a socket configured to receive the boss of the first housing. The second component may be configured to be detachably coupled to the first component by positioning the socket over the boss and rotating the second component into the recessed portion of the first component.

### CROSS-REFERENCE TO RELATED APPLICATIONS

This application claims the benefit of priority to U.S. Provisional Application No. 63/712,317, filed Oct. 25, 2024, which is incorporated herein by reference in its entirety.

### TECHNICAL FIELD

This disclosure is related to infusion devices, such as infusion devices comprising two or more components.

### BACKGROUND

Infusion devices are used to control delivery of medication directly into a patient's bloodstream. In some cases, the delivery of medication from an infusion device is slow and continuous. This can be useful, for example, to maintain a steady concentration of medication in a patient's bloodstream over many hours or days. Infusion devices can also respond quickly (e.g., in near real time) to biometric information from associated sensors. A common use for infusion devices is in the treatment of diabetes. Many patients with diabetes benefit from frequent or continuous infusion of insulin. This need can be temporary, such as during a hospital admission, or long-term. In the latter case, wearable infusion devices can enable a patient to receive necessary insulin infusion therapy while maintaining an active lifestyle. Wearable infusion devices typically include a battery-powered pump, a reservoir, and a cannula. The cannula is maintained in a transcutaneous position at an infusion site, such as under an adhesive patch applied directly to a patient's skin. In some cases, the pump and reservoir are in a separate unit flexibly tethered to the cannula. This unit, for example, can be strapped to a patient or attached to a patient's clothing. Alternatively, the pump and reservoir can be integrated with the cannula into a single unit configured to be directly attached to a patient's skin at an infusion site.

Without modern infusion devices, management of diabetes and other conditions that require continuous or frequent infusion of medication would be far more problematic. For example, many patients suffering from diabetes have difficulty actively monitoring their need for insulin (e.g., by performing blood glucose tests) and responding to this need appropriately (e.g., by self-administering appropriate quantities of insulin). Without assistance, these patients can be subject to a significant, ongoing risk of serious complications including diabetic coma and death. Even when active disease management is within a patient's capabilities, it can be time-consuming and stressful. Infusion devices, therefore, not only save lives, but they also meaningfully improve the quality of life for patients with diabetes and other diseases. Given that diabetes affects hundreds of millions of people worldwide and is only one example of a disease that often requires continuous or frequent infusion of medication, the importance of infusion devices is difficult to overstate. Accordingly, there is an ongoing need for improvement of these devices. Even small improvements in this field can have major public health benefits.

### SUMMARY

The present technology may be directed towards an infusion device comprising a first component and a second component configured to be detachably coupled to the first component. The first component can comprise a first housing, and in some embodiments the first housing defines a recessed portion and includes a boss. The second component can comprise a second housing configured to be received within the recessed portion of the first housing while the first and second components are detachably coupled to one another. In some embodiments, the second housing defines a socket configured to receive the boss of the first housing. The second component can be configured to be detachably coupled to the first component by positioning the socket over the boss and rotating the second component into the recessed portion of the first component.

Some embodiments of the present technology include a method for operating a medical device, where the method includes connecting a first component of the medical device and a second component of the medical device to one another. Connecting the first and second components to one another can include positioning the second component over a top side of the first component such that a socket of the second component is aligned with a boss of the first component, and such that a length axis of the second component is angled relative to a length axis of the first component. While the second component is angled relative to the first component, the method may further include moving the second component downwardly such that the socket is positioned over the boss. In some examples the method further includes rotating the second component towards the first component such that the respective length axes of the first and second components are substantially parallel.

According to several embodiments, the present technology includes an infusion device comprising a top surface, a bottom surface, and a height measured therebetween. The bottom surface may be configured to be positioned on a patient's skin during an infusion operation. The infusion device can include a first component and a second component configured to be detachably coupled to the first component. The first component can include a first housing defining a recessed portion and having a first retaining element, and the second component can include a second housing configured to be received within the recessed portion of the first housing while the first and second components are detachably coupled to one another. In some embodiments, the second component further comprises a second retaining element. The first and second retaining elements may be configured to engage one another when the first and second components are detachably coupled to constrain relative movement of the first and second components along a height dimension.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B are top and bottom perspective views, respectively, of a medical device in accordance with several embodiments of the present technology.

FIGS. 2A and 2B are bottom and top perspective views, respectively, of the medical device of FIG. 1 in a first stage of a coupling process, in accordance with several embodiments of the present technology.

FIGS. 2C and 2D are bottom perspective and top views, respectively, of the medical device of FIG. 1 in a second stage of a coupling process, in accordance with several embodiments of the present technology.

FIG. 3 is a top view of the medical device of FIG. 1 with the second component shown transparent, in accordance with several embodiments of the present technology.

FIGS. 4A and 4B are top and bottom perspective views, respectively, of the first component of the medical device shown in FIG. 1, in accordance with several embodiments of the present technology.

FIG. 4C is an enlarged front view of an arm of the first component shown in FIGS. 4A and 4B, in accordance with several embodiments of the present technology.

FIGS. 5A and 5B are side and bottom perspective views, respectively, of the second component of the medical device shown in FIG. 1, in accordance with several embodiments of the present technology.

FIG. 6 shows the arm of the first component of FIGS. 1A and 1B positioned in a recess of the second component, in accordance with several embodiments of the present technology.

FIG. 7 is an enlarged cross-sectional view of an anchor region, taken where indicated in FIG. 3.

FIG. 8 is an enlarged cross-sectional view of an anchor region, taken where indicated in FIG. 3.

FIG. 9 is an enlarged cross-sectional view of an anchor region, taken where indicated in FIG. 3.

FIG. 10 is a bottom perspective view of the medical device of FIG. 1, in accordance with several embodiments of the present technology.

FIG. 11 shows a cross-section of the arm of the first component positioned within the recess of the second component, in accordance with several embodiments of the present technology.

FIGS. 12A, 12B, 13A, and 13B illustrate a method of removing the second component from the first component, in accordance with several embodiments of the present technology.

### DETAILED DESCRIPTION

Devices in accordance with at least some embodiments of the present technology include innovative features that facilitate attaching components of the devices to one another and detaching the components from one another as needed. For example, the attaching and detaching processes can be simple and intuitive to facilitate performance of these operations by patients with little or no instructional guidance. Furthermore, when attached, the connection between the components can be secure enough to prevent the components from detaching from one another during use, such as during exercise or other activities that may subject the device to unpredictable forces.

Features that facilitate attaching and detaching components of wearable medical devices can be especially useful in the context of wearable infusion devices, such as those used to deliver insulin solution directly into a patient's bloodstream. In these and certain other devices, it can be useful for some elements to be more durable than other elements. For example, it can be useful for some elements to be multi-use and for other elements to be single-use (e.g., disposable). As a particular example, it is generally recommended to use a new cannula with every new infusion site to reduce the risk of infection. Moreover, the reservoir of infusion liquid in an infusion device is sometimes nonrefillable and, therefore, only usable for a limited period of time before it becomes depleted. In contrast, the pump motor, power source, wireless receiver, and other electronics are typically more compatible with reuse. These elements also tend to be more expensive than the cannula and the reservoir, such that replacing them every time a new infusion site is created would likely be cost prohibitive. Accordingly, it can be useful to incorporate the cannula, reservoir, and/or other elements most compatible with single-use into one component of an infusion device and to incorporate the pump motor, power source, wireless communication elements, other electronics, and/or other elements most compatible with multi-use into another component of the infusion device. This division can also facilitate protecting liquid-sensitive elements from exposure to infusion liquid. For these and/or other reasons, an intuitive mechanism for attaching and detaching components of infusion devices has the potential to reduce costs and improve usability, thereby expanding access to such devices for long-term use.

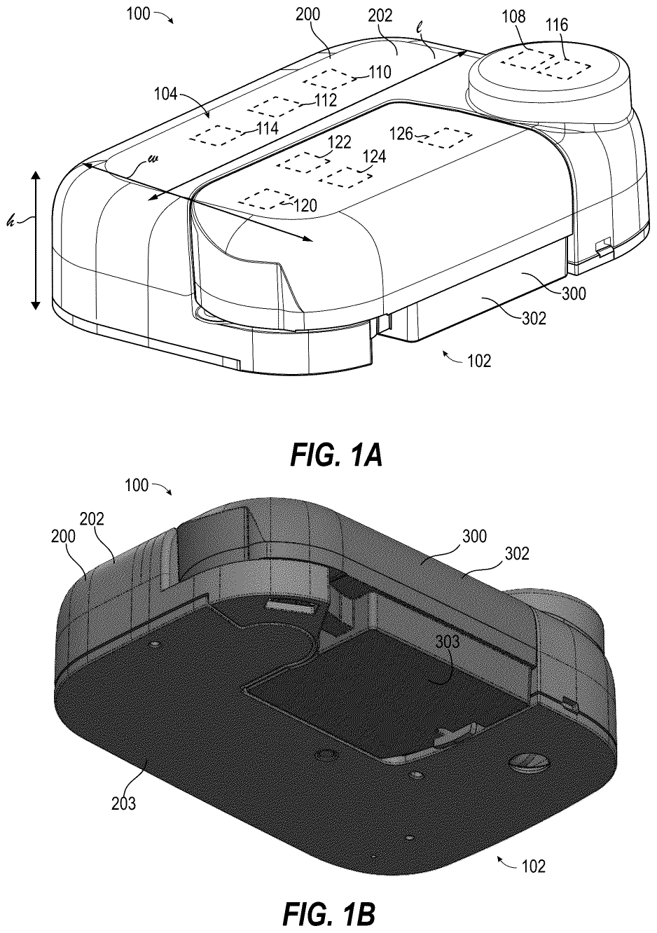

FIGS. 1A and 1B are top and bottom perspective views of a medical device 100 (or “device 100”) in accordance with at least some embodiments of the present technology. The device 100 comprises a first component 200 and a second component 300 configured to be detachably coupled to one another. In the depicted embodiment, the first component 200 is a disposable component and the second component 300 is a reusable component. In other embodiments, the first component 200 may be the reusable component and the second component 300 may be the disposable component. The device 100 has a bottom side 102 and a top side 104 opposite the bottom side 102 along a height dimension h of the device 100 (only labeled in FIG. 1A). The device 100 further includes a width dimension w and a length dimension 1 extending orthogonal to the width dimension w. The device 100 may be longer than it is wide (as shown in FIGS. 1A and 1B), wider than it is long, or have a width that is substantially the same as the length 1.

The first component 200 can include a first housing 202 having a first contact surface 203 (FIG. 1B) at the bottom side 102 of the device 100, where the first contact surface 203 is configured to be adhered to skin of a user while the patient wears the device 100. Similarly, the second component 300 can include a second housing 302 having a second contact surface 303 (FIG. 1B) at the bottom side 102, where the second contact surface 303 is configured to be positioned on or adjacent the user's skin while the patient wears the device 100. As best shown in FIG. 1B, the first and second contact surfaces 203, 303 can be in substantially the same plane when the first and second components 200, 300 are detachably connected to one another. In some embodiments, the first and second contact surfaces 203, 303 are positioned to both engage a single adhesive patch (not shown) when the first and second components 200, 300 are detachably coupled to one another. In other embodiments, only the first contact surface 203 or only the second contact surface 303 may engage an adhesive patch when the first and second components 200, 300 are detachably coupled to one another. This way, the component not adhered can be freely detached from the adhered component (and thus the user) while the adhered component remains on the user's skin. In several of such embodiments, the adhesive patch may be shaped to reflect a footprint of the component to which it is configured to be attached.

The first and second housings 202, 302 can enclose and support a plurality of internal components that enable subcutaneous delivery of a fluid medicament to a user while the user is wearing the device 100. For example, the first housing 202 can enclose and support a cannula 108 (shown schematically in FIG. 1A only), a reservoir 110 configured to hold a fluid medicament (e.g., an insulin solution), a plunger 112 disposed at least partially within the reservoir 110, and a drive assembly 114 operably coupled to the plunger 112 and configured to move the plunger 112 within the reservoir 110 to deliver the fluid medicament to the user via the cannula 108. In some cases, the reservoir 110 is refillable. In other cases, the reservoir 110 is not refillable. The cannula 108 can be extendable from a retracted position within the first housing 202 toward a transcutaneous extended position. The first housing 202 may further enclose and support an insertion assembly 116 configured to move the cannula 108 from the retracted position to the extended position via actuation by the user (e.g., via a trigger on the first housing 202 or via remote actuation). It will be appreciated that the first component 200 may include more or fewer internal components than those described above. For example, in some embodiments the first component 200 may not include the cannula 108, and/or the reservoir 110, and/or the plunger 112, and/or the drive assembly 114, and/or the insertion assembly 116.

The second housing 302 can enclose and support a motor 120, a controller 122, an energy source 124, and a wireless receiver 126 configured to be in communication with the controller 122 and an external device (not shown). The controller 122 can be configured to cause fluid medicament from the reservoir 110 to flow to the cannula 108 at least partially in response to a signal received by the wireless receiver 126. For example, the wireless receiver 126 can receive a signal indicating that a patient's blood-glucose is rising. Based on this signal alone or in combination with other information, the controller 122 can instruct the motor 120 to energize the drive assembly 114 to move the plunger 112 to deliver the fluid medicament from the reservoir 110 into the patient's bloodstream via the cannula 108. The signal can be based on biometric data, user input, and/or other information from an external device in wired or wireless communication with the device 100. The external device, for example, can be a sensor, such as a continuous glucose monitor, and/or a computer, such as smart phone or specialized external remote with data input by a user. It will be appreciated that the second component 300 may include more or fewer internal components than those described above. For example, in some embodiments the second component 300 may not include the motor 120, and/or the controller 122, and/or the energy source 124, and/or the wireless receiver 126.

In some embodiments, the device 100 includes a third component (not shown) which may include any of the foregoing internal components mentioned above. The third component may be configured to detachably couple to one or both of the first and second components 200, 300, and/or may be configured to fixedly couple to one of the first or second components 200, 300.

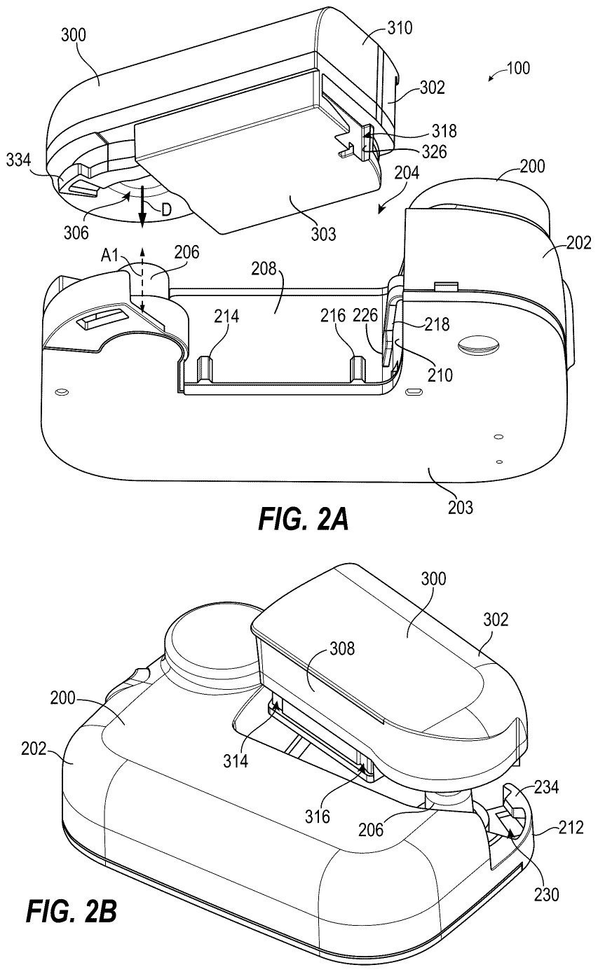

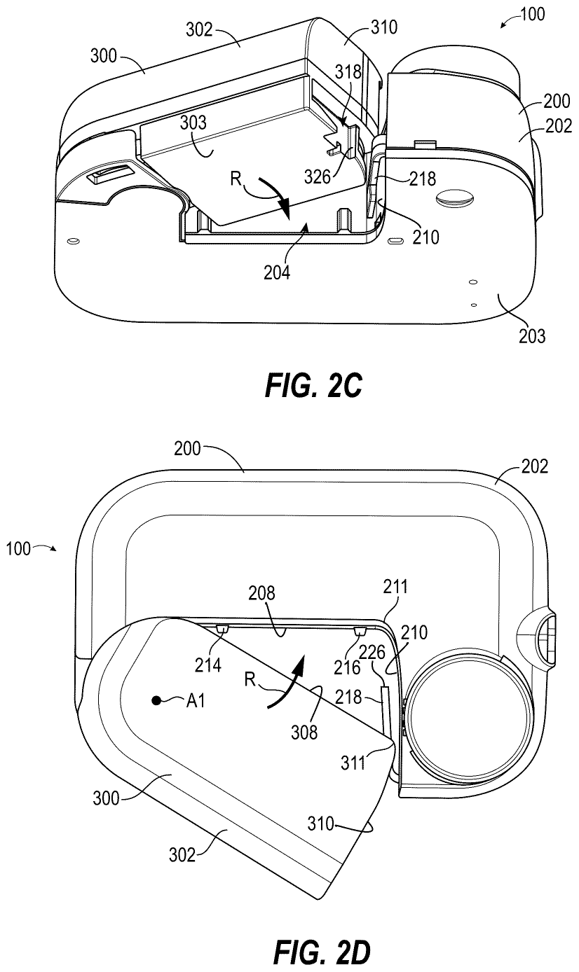

FIG. 2A is a bottom perspective view of the device 100 in a decoupled state, and FIGS. 2A-2C depict a method for coupling the second component 300 to the first component 200. As shown, the first housing 202 comprises first and second engagement walls 208, 210 that come together at inside corner 211 (FIG. 2C) and define a recessed portion 204 of the first housing 202. The recessed portion 204 is configured to receive the second component 300 when the first and second components 200, 300 are detachably coupled to one another. The second housing 302 comprises first and second engagement walls 308, 310 (308 only visible in FIG. 2B) that meet at outside corner 311 (FIG. 2D) and are configured to be positioned parallel and adjacent to the first and second engagement walls 208, 210, respectively, of the first housing 202 when the first and second components 200, 300 are detachably coupled to one another.

The first and second housings 202, 302 may further include a mating boss 206 and socket 306, respectively, and an arm 218 and recess 318, respectively. The boss 206 is configured to be positioned within the socket 306 and the arm 218 within the recess 318 when the first and second components 200, 300 are detachably coupled to one another. Engagement of these structures, as well as apposition of the first and second engagement walls 208, 308 and 210, 310, respectively, constrain movement of the second component 300 relative to the first component 200 along a width dimension and a length dimension, as detailed below. It will be appreciated that the boss 206 and socket 306 can be reversed (e.g., the second housing 302 includes the boss and the first housing 202 includes the socket) or may comprise other complementary engagement structures, such as a first engagement structure and a second engagement structure configured to couple to one another along a height dimension.

The second component 300 can be coupled to the first component 200 via a two-step engagement process, beginning with positioning the socket 306 over the boss 206, as shown in FIG. 2A. The user may position the second component 300 above a top side of the first component 200 with the second component 300 angled relative to the first component 200 (e.g., the first engagement wall 308 of the second housing 302 is disposed at an angle relative to the first engagement wall 208 of the first housing 202) and the socket 306 aligned with the boss 206. In this angled position, the second component 300 is then moved downwardly (depicted by arrow D) towards the first component 200 such that the socket 306 is positioned around the boss 206 and the contact surfaces 203, 303 of the first and second components 200, 300 are substantially within the same plane (as shown in FIG. 2C). FIG. 2D a top view of FIG. 2C. Next, the user may rotate the second component 300 about the boss axis A1 into the recessed portion 204 of the first housing 202, thereby bringing the first and second engagement walls 208, 308 and 210, 310, respectively, substantially parallel and into apposition with one another. As the second component 300 rotates into place, the second housing 302 bends the arm 218 towards the second engagement wall 210 until the arm 218 is aligned with the recess 318, at which point the arm 218 snaps outwardly into the recess 318 and engages a wall 326 within the recess 318 that prevents the second component 300 from rotating out of the recessed portion 204. Release and removal of the second component 300 from the first component 200 is discussed in greater detail below with reference to FIGS. 10-13B.

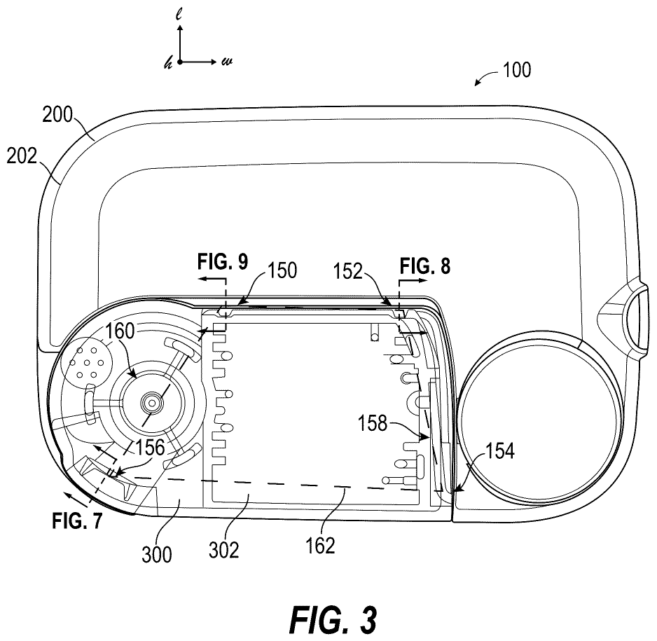

While the first and second components 200, 300 remain coupled to one another, several features prevent or limit relative movement. FIG. 3 is a top view of the assembled device 100 with the second component 300 transparent for case of viewing the constraining features. As shown, the device 100 includes a plurality of localized anchor regions (labeled 150, 152, 154, 156, 158, and 160) in which constraining elements from each of the first and second components 200, 300 engage one another to limit or prevent relative movement of the first and second components 200, 300 in the length 1, width w, and/or height h dimension of the device 100. Anchor region 160, for example, comprises the boss 206 of the first component 200 and socket 306 of the second component 300. Engagement of these two structures restricts relative movement of along a width w and length 1 dimension. It will be appreciated that, while not called out specifically, the apposing first engagement walls 208, 308 and second engagement walls 210, 310 also prevent relative movement along the length 1 and width w dimensions of the device 100.

One or more anchor regions may be realized by engagement between the arm 218 of the first component and features within the recess 318 of the second component 300. Anchor region 158, for example, may prevent or limit rotation of the second component 300 out of the recessed portion 204 (e.g., restricting movement along both the width w and length 1 dimensions), and anchor region 154 may prevent or limit relative movement between the first and second components along a height dimension h (perpendicular to the face of the page). Before discussing these and the other anchor regions, the relevant features of the first and second components 200, 300 will be described in more detail.

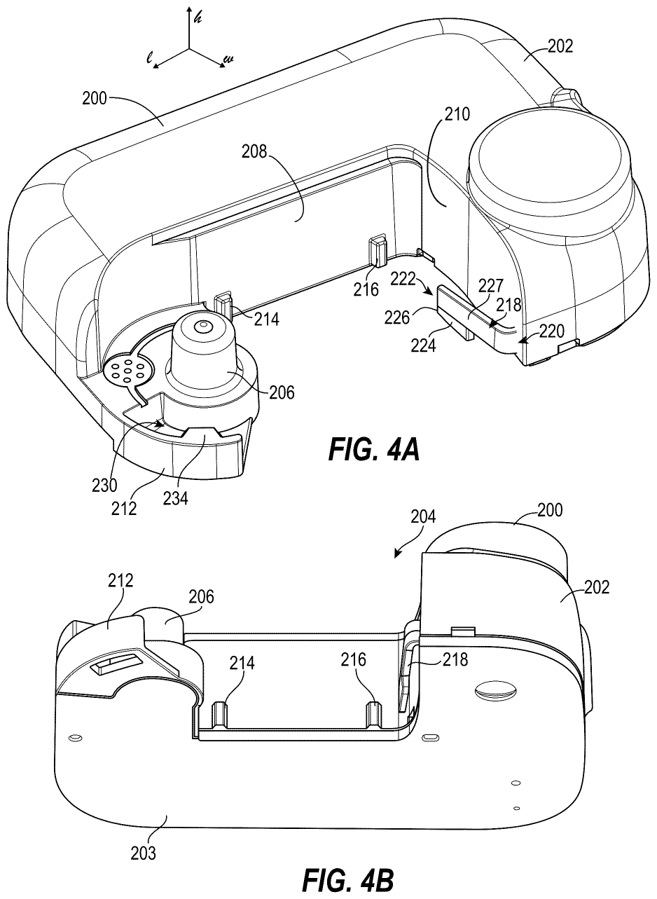

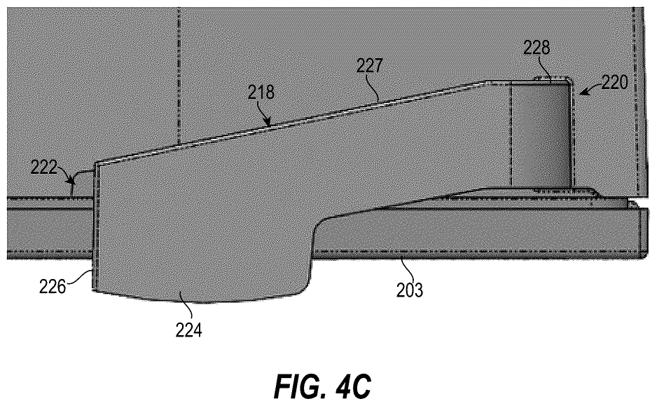

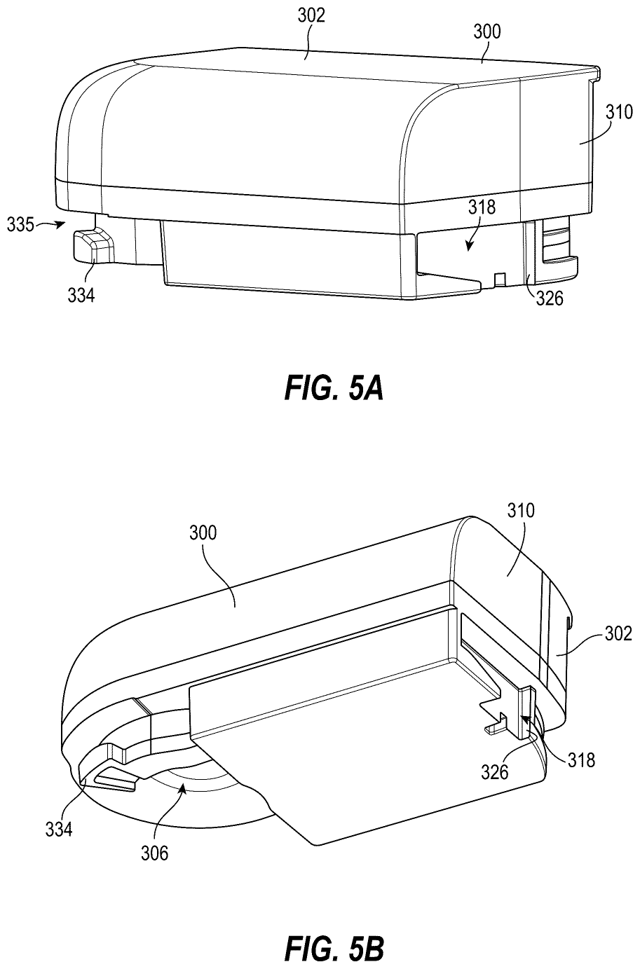

FIGS. 4A and 4B show top and bottom perspective views, respectively, of the first component 200, and FIG. 4C shows an enlarged front view of the arm 218. FIGS. 5A and 5B show side and bottom perspective views, respectively, of the second component 300. Referring to FIGS. 4A-5B together, the first housing 202 includes first and second engagement walls 208, 210 that are angled relative to one another and define two sides of the recessed portion 204. In some embodiments, the first and second engagement walls 208, 210 are substantially orthogonal to one another with the first engagement wall 208 extending along a length dimension 1 and the second engagement wall 210 extending along the width dimension w (or vice versa). In other embodiments the first and engagement second walls 208, 210 may be disposed at a non-90-degree angle relative to one another. Likewise, the first and second engagement walls 308, 310 of the second component 300 may be substantially orthogonal to one another with the first engagement wall 308 extending along a length dimension and the second engagement wall 310 extending along a width dimension (or vice versa). In other embodiments the first and second engagement walls 308, 310 may be disposed at a non-90-degree angle relative to one another. In any case, the angle between the first and second engagement walls 308, 310 may be substantially the same as that between the first and second engagement walls 208, 210 of the first housing 202, as the second component 300 is configured to fit snugly within the recessed portion 204 of the first component 200.

As previously discussed, the first component 200 may include an arm 218 extending away from the second engagement wall 210 towards the first engagement wall 208. The arm 218 may have a proximal end portion 220 at the second engagement wall 210 and a distal end portion 222 disposed within the recessed portion 204. When the arm 218 is in a resting position, not engaged with the second component 300, a distal end portion 222 of the arm 218 is spaced apart from the second engagement wall 210 by a distance measured along the length 1 dimension. The arm 218 may comprise a body 227 that extends between the proximal and distal end portions 220, 222, a handle 224 that extends downwardly from a distal portion of the body 227, and a distally facing surface 226. In some embodiments, the surface 226 faces towards the first engagement wall 208. The arm 218 may be integral with the first housing 202 (e.g., molded with the rest of the first housing 202) or may be a separate element coupled to the first housing 202 during assembly.

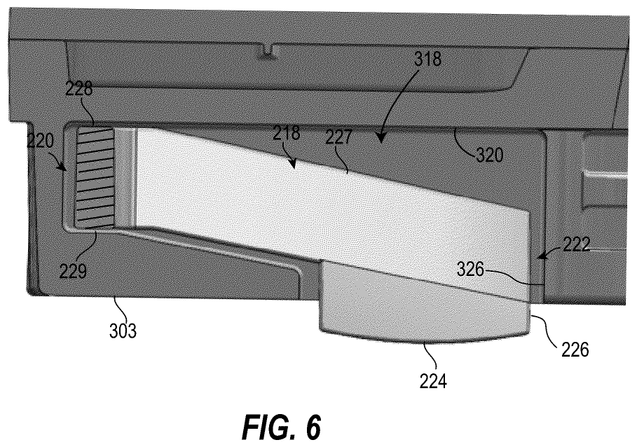

As best shown in FIG. 4C, the arm 218 may extend at a downward angle relative to a bottom contact surface 203 of the first component 200. This way, the body 227 of the arm 218 is spaced apart from the top surface 320 defining the recess 318 (see FIG. 6) when the arm 218 is positioned within the recess 318, which facilitates rotation of the arm 218 out of the recess 318, as described below with reference to FIGS. 10 and 11. The handle 224 may extend beyond the contact surface 203 which enables a user to manipulate the position of the arm 218 when the device 100 is assembled. Further details regarding manipulation of the arm 218 to release the second component 300 is detailed below with reference to FIGS. 10 and 11.

During the second stage of coupling in which the second component 300 is rotated inwardly towards the first component 200 (as shown in FIGS. 2C and 2D), the leading edge of the second component 300 bends the arm 218 towards the second engagement wall 210 of the first component 200. The arm 218 can be resilient such that, once the recess 318 is aligned with the arm 218, the arm 218 bends back outwardly, into the recess 318 (see FIG. 6). Once within the recess 318, the surface 226 at the distal portion of the arm 218 abuts a wall 326 within the recess 318, thereby forming an anchor region 154 (FIG. 3) that prevents the second component 300 from rotating away from the first component 200 and out of the recessed portion 204.

With reference to FIG. 6, the arm 218 and recess 318 may further form a second anchor region 158 (FIG. 3) at the interface between the top and bottom surfaces 228, 229 at the proximal end portion 220 of the arm 218 and the adjacent surfaces defining the top and bottom sides of the recess 318. The opposing surfaces may limit or prevent relative movement between the first and second components 200, 300 along a height dimension h of the device 100.

Referring again to FIGS. 4A-5B, the first housing 202 may include a ledge 212 extending away from a bottom region of the first engagement wall 208 in a direction substantially parallel to a width dimension w of the device 100. The boss 206 may extend upwardly from a top surface of the ledge 212. The ledge 212 may define a curved channel 230 that opens to a top surface of the ledge 212 and that is configured to receive a portion of the second housing 302 therein. The ledge 212 may further include a lip 234 that extends over a portion of the channel 230 and is configured to engage a protrusion 334 at a bottom side of the second component 300 to resist vertical movement of the second component 300 relative to the first component 200 when the second component 300 is detachably coupled to the first component 200.

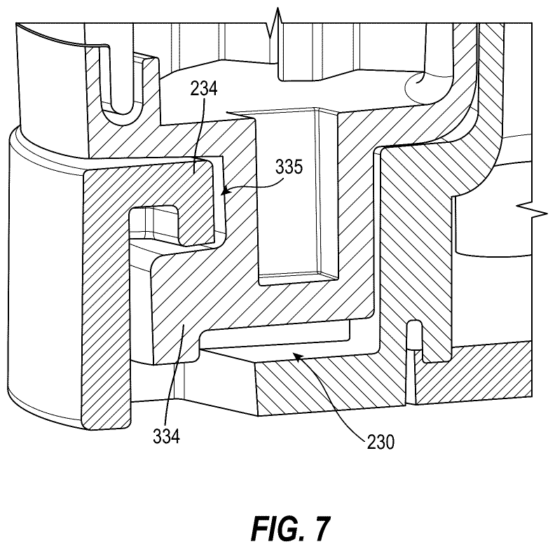

During the first stage of coupling in which the second component is moved downwardly over the boss 206, the protrusion 334 may be positioned in the channel 230 upstream of the lip 234 such that the lip 234 does not extend over the protrusion 334. As the second component 300 is rotated inwardly towards the first component 200, the protrusion 334 slides within the channel 230 to a position underneath the lip 234, which simultaneously positions the lip 234 within the channel 335 of the second housing 302. FIG. 7 shows a cross-section of the lip 234 sandwiched between the top and bottom surfaces of the channel 335 and the protrusion 334 constrained within the channel 230 under the lip 234. As such, the lip 234, protrusion 334, and channels 230, 335 together form an anchor region 156 (FIG. 3) that constrains relative movement along a height h dimension of the device 100.

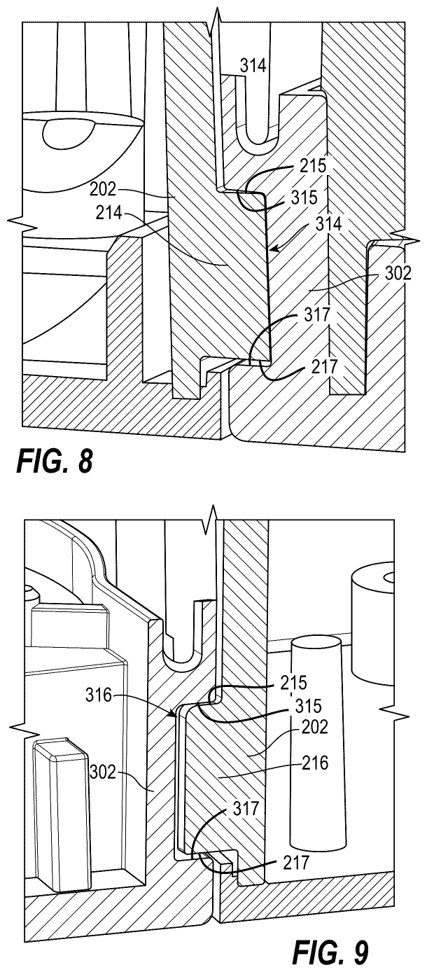

Referring again to FIGS. 4A-5B, the first engagement wall 208 of the first housing 202 can include one or more protrusions 214, 216 configured to be received within one or more corresponding pockets 314, 316 defined by the first engagement wall 308 of the second housing 302. The protrusions 214, 216 can be spaced apart along a length dimension 1 of the first engagement wall 208, and similarly the pockets 314, 316 can be spaced apart along a length dimension 1 of the first engagement wall 308. FIGS. 8 and 9 are cross-sectional views of the protrusions/pockets 214, 314 and 216, 316, respectively, while the first and second components 200, 300 are detachably coupled to one another. As shown, the portions 315, 317 of the first engagement wall 308 bounding the top and bottom sides of the pockets 314, 316 engage the top and bottom surfaces 215, 217 of the protrusions 214, 216 to resist vertical movement of the protrusions 214, 216 (and thus the first component 200) relative to the second component 300. Accordingly, each pair of protrusions 214, 216 and pockets 314, 316 form a respective anchor region 150, 152 (FIG. 3) that constrains relative movement along a height h dimension of the device 100.

While the drawings show two protrusions along the first engagement wall 208, the first component 200 may include a single protrusion or more than two protrusions. Likewise, while the drawings show two pockets along the first engagement wall 308, the second component 300 may include a single pocket or more than two pockets.

The device 100 can therefore having a plurality of anchor regions configured to constrain relative movement of the first and second components 200, 300 relative to one another along a height dimension h when the first and second components 200, 300 are detachably coupled to one another. As best visualized in FIG. 3, the height-restricting anchor regions 150, 152, 154, 156 can be distributed about the interface between the first and second components 200, 300 to enclose as great a footprint as possible, thus adding stability. The perimeter 162 of the area enclosed by the connected anchor regions may also be designed to cross or pass near the boss 206, or axis of rotation of the second component 300, thereby further adding stability. While some degree of securement between the first and second components 200, 300 may prevent premature detachment, it may be desirable to further stabilize the connection with additional anchor regions, as any play or “wiggle” between the two components may be uncomfortable or distracting for the user. The device 100 may have two, three, four, five, six, seven, eight or more such anchor regions.

FIG. 10 is a bottom perspective view of the device 100 in an assembled state. Similar to the method of coupling the second component 300 to the first component 200, removal of the second component 300 from the first component 200 occurs in two stages. To begin, the user may engage the handle 224 (protruding from the contact surfaces 203, 303 of the first and second housings 202, 302) with a single finger (or thumb) and push the distal portion of the arm 218 towards the second engagement wall 210 of the first housing 202 (as indicated by arrow A1). In so doing, and as depicted in FIG. 11, the surface 226 of the arm 218 slides laterally off the wall 326 within the recess 318 of the second housing 302, which then enables the user to rotate the second component 300 outwardly (as indicated by arrow A2 in FIG. 10), away from the first engagement wall 208. As previously mentioned, the arm 218 extends downwardly within the recess 318 such that a gap g remains between a top surface of the body 227 of the arm 218 and a top surface 320 defining the recess 318. As such, when the handle 224 is deflected by the user, the entire height h of the distal portion of the arm 218 translates with it and fully clears the top surface 320 of the recess 318. Without the relief, the arm 218 could twist and the upper portion of the distal portion could remain engaged with the top surface 320.

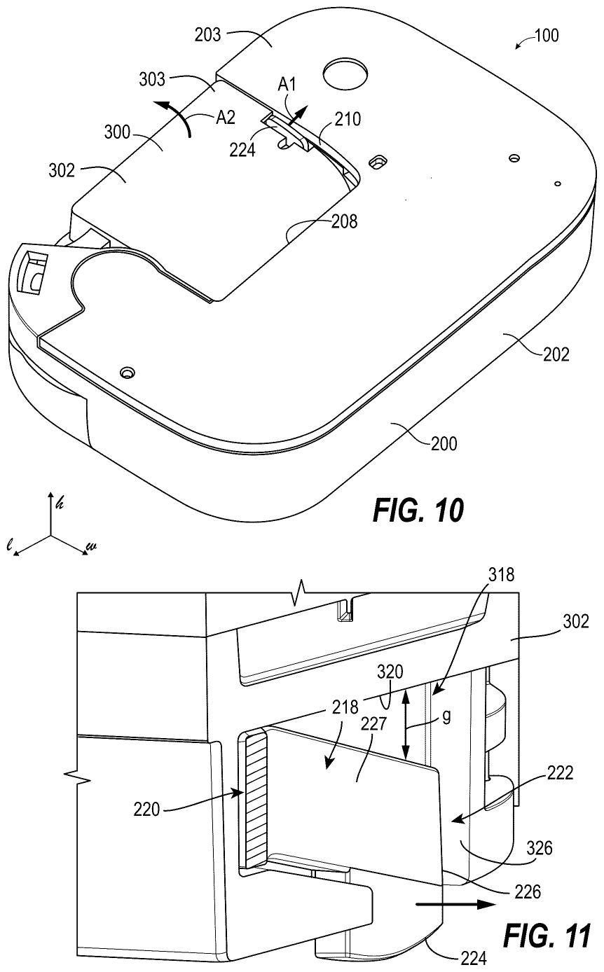

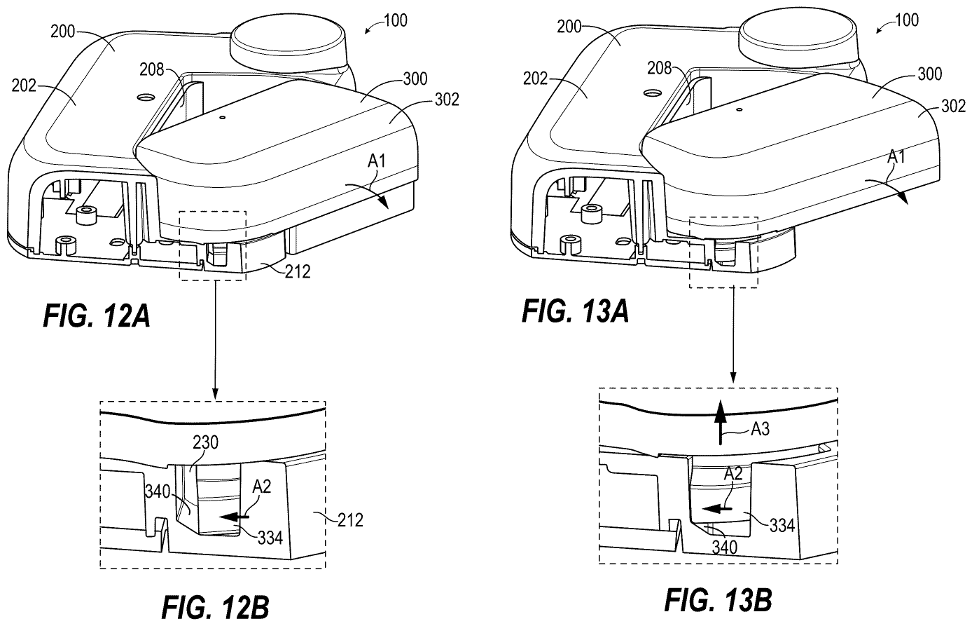

In some embodiments, the device 100 may optionally include one or more features to facilitate removal of the boss 206 from the socket 306 (see FIGS. 4A-5B), as the two components may be shaped to have a tight fit. As shown in FIGS. 12A-13B, the floor of the first housing 202 defining the channel 230 may have a ramp 340 at one end. As the second component 300 is rotated to disconnect (indicated by arrow A1), the protrusion 334 on the second housing 302 slides within the channel 230 (indicated by arrow A2) and is forced onto the ramp 340. Sliding of the protrusion 334 along the ramp 340 pushes the second component 300 upwardly (indicated by arrow A3 in FIG. 13A) to help separate the socket 306 from the boss 206. In some embodiments, the device 100 does not include the ramp 340.

### EXAMPLES

Various examples of aspects of the subject technology are described as numbered examples (1, 2, 3, etc.) for convenience. These are provided as examples and do not limit the subject technology.

1. An infusion device, comprising:
a first component comprising a first housing, the first housing defining a recessed portion and including a boss; and
a second component configured to be detachably connected to the first component, wherein the second component comprises a second housing and the second housing is configured to be received within the recessed portion of the first housing while the first and second components are detachably coupled to one another, the second housing defining a socket configured to receive the boss of the first housing,
wherein the second component is configured to be detachably coupled to the first component by positioning the socket over the boss and rotating the second component into the recessed portion of the first component.

2. The infusion device of Example 1, wherein the first housing comprises an arm and the second housing defines a recess configured to receive the arm when the first and second components are detachably coupled to one another.

3. The infusion device of Example 2, wherein the arm is configured to engage a surface of the second housing within the recess to prevent rotation of the second component out of the recessed portion of the first housing.

4. The infusion device of Example 2 or Example 3, wherein the arm is configured to be manipulated by a user to move the arm out of the recess, thereby enabling rotation of the second component out of the recessed portion of the first component.

5. The infusion device of any one of Examples 2 to 4, wherein the arm comprises a proximal end portion, a distal end portion, and a handle extending downwardly from the distal end portion, and wherein, when the first and second components are detachably coupled to one another, the handle protrudes beyond a bottom surface of the infusion device so that the arm may be manipulated by a user.

6. The infusion device of any one of Examples 2 to 5, wherein the arm comprises a proximal end portion and a distal end portion, and wherein the arm extends downwardly within the recess such that a top surface at the distal end portion of the arm is spaced farther from a top wall defining the recess than a top surface at the proximal end portion of the arm.

7. The infusion device of any one of Examples 1 to 6, wherein:
the recessed portion of the first housing is defined by first and second engagement walls, and wherein the second housing comprises first and second engagement walls,
prior to rotation of the second housing into the recessed portion, the first engagement walls of the first and second housings are angled relative to one another and the second engagement walls of the first and second housings are angle relative to one another, and
after rotation of the second housing into the recessed portion, the first engagement walls are substantially parallel to one another and the second engagement walls are substantially parallel to one another.

8. The infusion device of Example 7, wherein the first housing includes a deformable arm extending away from the second engagement wall and the second housing includes a recess defined by the second engagement wall, and wherein the arm is configured to be received within the recess when the first and second components are detachably coupled to one another.

9. The infusion device of any one of Examples 1 to 8, wherein:
the first component includes a cannula, a reservoir configured to contain a fluid medicament, a plunger at least partially disposed within the reservoir, and a drive assembly operably coupled to the plunger,
the second component includes an energy source configured to be operably coupled to the drive assembly, and
wherein activation of the drive assembly by the energy source causes the plunger to move within the reservoir to dispel fluid medicament from the reservoir into the cannula.

10. A method for operating a medical device, the method comprising:
connecting a first component of the medical device and a second component of the medical device to one another, wherein connecting the first and second components to one another includes:
   positioning the second component over a top side of the first component such that a socket of the second component is aligned with a boss of the first component, and such that a length axis of the second component is angled relative to a length axis of the first component,
   with the second component angled relative to the first component, moving the second component downwardly such that the socket is positioned over the boss, and
   rotating the second component towards the first component such that the respective length axes of the first and second components are substantially parallel.

11. The method of Example 10, wherein connecting the first and second components further includes positioning an arm of the first component within a recess of the second component as the second component is rotated towards the first component.

12. The method of Example 10 or Example 11, further comprising:
disconnecting the first and second components from one another, wherein disconnecting the first and second components from one another includes:
   moving at least a portion of the arm of the first component out of the recess of the second component, and
   after moving the arm, rotating the second component away from the first component, and
   lifting the second component off the first component to disengage the boss and the socket.

13. An infusion device, comprising:
a top surface, a bottom surface, and a height measured therebetween, wherein the bottom surface is configured to be positioned on a patient's skin during an infusion operation;
a first component including a first housing, the first housing defining a recessed portion and having a first retaining element; and
a second component configured to be detachably connected to the first component, wherein the second component includes a second housing configured to be received within the recessed portion of the first housing while the first and second components are detachably coupled to one another, the second component further comprising a second retaining element,
wherein the first and second retaining elements are configured to engage one another when the first and second components are detachably coupled to constrain relative movement of the first and second components along a height dimension.

14. The infusion device of Example 13, wherein the first and second retaining elements together comprise an anchor region, and wherein the infusion device includes a plurality of anchor regions.

15. The infusion device of Example 13 or Example 14, wherein the first housing comprises an arm and the second housing defines a recess configured to receive the arm when the first and second components are detachably coupled to one another.

16. The infusion device of Example 15, wherein the arm is configured to engage a surface of the second housing within the recess to prevent rotation of the second component out of the recessed portion of the first housing.

17. The infusion device of Example 15 or Example 16, wherein the arm is configured to be manipulated by a user to move the arm out of the recess, thereby enabling rotation of the second component out of the recessed portion of the first component.

18. The infusion device of any one of Examples 15 to 17, wherein the arm comprises a proximal end portion, a distal end portion, and a handle extending downwardly from the distal end portion, and wherein, when the first and second components are detachably coupled to one another, the handle protrudes beyond a bottom surface of the infusion device so that the arm may be manipulated by a user.

19. The infusion device of any one of Examples 15 to 18, wherein the arm comprises a proximal end portion and a distal end portion, and wherein the arm extends downwardly within the recess such that a top surface at the distal end portion of the arm is spaced farther from a top wall defining the recess than a top surface at the proximal end portion of the arm.

20 The infusion device of any one of Examples 13 to 19, wherein:
the first component includes a cannula, a reservoir configured to contain a fluid medicament, a plunger at least partially disposed within the reservoir, and a drive assembly operably coupled to the plunger, and
the second component includes an energy source configured to be operably coupled to the drive assembly,
wherein activation of the drive assembly by the energy source causes the plunger to move within the reservoir to dispel fluid medicament from the reservoir into the cannula.

21. An infusion device, comprising:
a first component comprising a first housing, the first housing defining a recessed portion and including a first engagement structure; and
a second component configured to be detachably connected to the first component, wherein the second component comprises a second housing and the second housing is configured to be received within the recessed portion of the first housing while the first and second components are detachably coupled to one another, the second housing defining a second engagement structure configured to detachably engage and/or couple to the first engagement structure of the first housing,
wherein the second component is configured to be detachably coupled to the first component by positioning the second engagement structure over the first engagement structure and rotating the second component into the recessed portion of the first component.

22. An infusion device, comprising:
a top surface, a bottom surface, and a height measured therebetween, wherein the bottom surface is configured to be positioned on a patient's skin during an infusion operation;
a first component including a first housing, the first housing defining a recessed portion and having a first retaining element; and
a second component configured to be detachably connected to the first component, wherein the second component includes a second housing configured to be received within the recessed portion of the first housing while the first and second components are detachably coupled to one another, the second component further comprising a second retaining element,
wherein the first and second retaining elements are configured to engage one another when the first and second components are detachably coupled to constrain relative movement of the first and second components along a height dimension.

### CONCLUSION

Although many of the embodiments are described above with respect to systems, devices, and methods for detaching components of wearable infusion pumps, the technology is applicable to other applications and/or other approaches, such as non-wearable medical devices or two-part devices that are not infusion pumps. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-13B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An infusion device, comprising:
a first component comprising a first housing, the first housing defining a recessed portion and including a boss; and
a second component configured to be detachably connected to the first component, wherein the second component comprises a second housing and the second housing is configured to be received within the recessed portion of the first housing while the first and second components are detachably coupled to one another, the second housing defining a socket configured to receive the boss of the first housing,
wherein the second component is configured to be detachably coupled to the first component by positioning the socket over the boss and rotating the second component into the recessed portion of the first component.

2. The infusion device of claim 1, wherein the first housing comprises an arm and the second housing defines a recess configured to receive the arm when the first and second components are detachably coupled to one another.

3. The infusion device of claim 2, wherein the arm is configured to engage a surface of the second housing within the recess to prevent rotation of the second component out of the recessed portion of the first housing.

4. The infusion device of claim 2, wherein the arm is configured to be manipulated by a user to move the arm out of the recess, thereby enabling rotation of the second component out of the recessed portion of the first component.

5. The infusion device of claim 2, wherein the arm comprises a proximal end portion, a distal end portion, and a handle extending downwardly from the distal end portion, and wherein, when the first and second components are detachably coupled to one another, the handle protrudes beyond a bottom surface of the infusion device so that the arm may be manipulated by a user.

6. The infusion device of claim 2, wherein the arm comprises a proximal end portion and a distal end portion, and wherein the arm extends downwardly within the recess such that a top surface at the distal end portion of the arm is spaced farther from a top wall defining the recess than a top surface at the proximal end portion of the arm.

7. The infusion device of claim 1, wherein:
the recessed portion of the first housing is defined by first and second engagement walls, and wherein the second housing comprises first and second engagement walls,
prior to rotation of the second housing into the recessed portion, the first engagement walls of the first and second housings are angled relative to one another and the second engagement walls of the first and second housings are angle relative to one another, and
after rotation of the second housing into the recessed portion, the first engagement walls are substantially parallel to one another and the second engagement walls are substantially parallel to one another.

8. The infusion device of claim 7, wherein the first housing includes a deformable arm extending away from the second engagement wall and the second housing includes a recess defined by the second engagement wall, and wherein the arm is configured to be received within the recess when the first and second components are detachably coupled to one another.

9. The infusion device of claim 1, wherein:
the first component includes a cannula, a reservoir configured to contain a fluid medicament, a plunger at least partially disposed within the reservoir, and a drive assembly operably coupled to the plunger,
the second component includes an energy source configured to be operably coupled to the drive assembly, and
wherein activation of the drive assembly by the energy source causes the plunger to move within the reservoir to dispel fluid medicament from the reservoir into the cannula.

10. A method for operating a medical device, the method comprising:
connecting a first component of the medical device and a second component of the medical device to one another, wherein connecting the first and second components to one another includes:
positioning the second component over a top side of the first component such that a socket of the second component is aligned with a boss of the first component, and such that a length axis of the second component is angled relative to a length axis of the first component,
with the second component angled relative to the first component, moving the second component downwardly such that the socket is positioned over the boss, and
rotating the second component towards the first component such that the respective length axes of the first and second components are substantially parallel.

11. The method of claim 10, wherein connecting the first and second components further includes positioning an arm of the first component within a recess of the second component as the second component is rotated towards the first component.

12. The method of claim 11, further comprising:
disconnecting the first and second components from one another, wherein disconnecting the first and second components from one another includes:
moving at least a portion of the arm of the first component out of the recess of the second component, and
after moving the arm, rotating the second component away from the first component, and
lifting the second component off the first component to disengage the boss and the socket.

13. An infusion device, comprising:
a top surface, a bottom surface, and a height measured therebetween, wherein the bottom surface is configured to be positioned on a patient's skin during an infusion operation;
a first component including a first housing, the first housing defining a recessed portion and having a first retaining element; and
a second component configured to be detachably connected to the first component, wherein the second component includes a second housing configured to be received within the recessed portion of the first housing while the first and second components are detachably coupled to one another, the second component further comprising a second retaining element,
wherein the first and second retaining elements are configured to engage one another when the first and second components are detachably coupled to constrain relative movement of the first and second components along a height dimension.

14. The infusion device of claim 13, wherein the first and second retaining elements together comprise an anchor region, and wherein the infusion device includes a plurality of anchor regions.

15. The infusion device of claim 13, wherein the first housing comprises an arm and the second housing defines a recess configured to receive the arm when the first and second components are detachably coupled to one another.

16. The infusion device of claim 15, wherein the arm is configured to engage a surface of the second housing within the recess to prevent rotation of the second component out of the recessed portion of the first housing.

17. The infusion device of claim 15, wherein the arm is configured to be manipulated by a user to move the arm out of the recess, thereby enabling rotation of the second component out of the recessed portion of the first component.

18. The infusion device of claim 15, wherein the arm comprises a proximal end portion, a distal end portion, and a handle extending downwardly from the distal end portion, and wherein, when the first and second components are detachably coupled to one another, the handle protrudes beyond a bottom surface of the infusion device so that the arm may be manipulated by a user.

19. The infusion device of claim 15, wherein the arm comprises a proximal end portion and a distal end portion, and wherein the arm extends downwardly within the recess such that a top surface at the distal end portion of the arm is spaced farther from a top wall defining the recess than a top surface at the proximal end portion of the arm.

20. The infusion device of claim 13, wherein:
the first component includes a cannula, a reservoir configured to contain a fluid medicament, a plunger at least partially disposed within the reservoir, and a drive assembly operably coupled to the plunger, and
the second component includes an energy source configured to be operably coupled to the drive assembly,
wherein activation of the drive assembly by the energy source causes the plunger to move within the reservoir to dispel fluid medicament from the reservoir into the cannula.
